(19) United States
(12) Patent Application Publication
(10) Pub. No.: US 2023/0126384 A1
(43) Pub. Date: Apr. 27, 2023

(54) METHODS OF VISUALIZATION OF STAINS ON FABRIC

(71) Applicant: Church & Dwight Co., Inc., Princeton, NJ (US)

(72) Inventors: Steven T. Adamy, Lawrenceville, NJ (US); Andrew Guttentag, Robbinsville, NJ (US); Micki Brain, Flemington, NJ (US)

(73) Assignee: Church & Dwight Co., Inc., Princeton, NJ (US)

(21) Appl. No.: 17/972,847

(22) Filed: Oct. 25, 2022

Related U.S. Application Data
(60) Provisional application No. 63/271,935, filed on Oct. 26, 2021.

(51) Int. Cl. G01N 21/64; D06H 3/08

(52) U.S. Cl. CPC G01N 21/6428; D06H 3/08; G01N 2021/6439

(57) ABSTRACT

The present disclosure provides methods for visualizing a soiling substance on a fabric and, in some aspects, methods of quantifying the efficacy of a composition for removal of a soiling substance from a fabric. In some aspects, the disclosure provides methods for visualizing a soiling substance on a fabric which include providing a fabric that includes at least one soiling substance, applying a signaling agent to an area of the fabric that includes the at least one soiling substance, illuminating at least a portion of the area of the fabric that includes the at least one soiling substance and the signaling agent with light of a wavelength or wavelength band that is one or both of absorbed by the signaling agent or effective to cause fluorescent emission from the signaling agent, detecting one or both of an absorbance signal and an emission signal from the signaling agent when illuminated.

### CROSS-REFERENCE TO RELATED APPLICATIONS

The present application claims priority to U.S. Provisional Patent Application No. 63/271,935, filed Oct. 26, 2021, the disclosure of which is incorporated by reference in its entirety.

### FIELD OF THE DISCLOSURE

The present disclosure relates to methods of visualization and measurement of stains or soiling substances on a fabric and methods for quantifying the efficacy of various compositions for removal of stains on a fabric.

### BACKGROUND

Many stains and soils are either colorless or lack sufficient color to clearly see or make meaningful reflectance measurements using conventional imaging systems, e.g., which use colorimetric and/or visible spectra. Typically, one way this challenge is addressed is by adding a colored dye or pigment to the stain or soil, which can be used as an indicator for stain removal. Stained fabrics are then imaged and analyzed before and after washing. Commonly, reflectance values are recorded by a spectrophotometer to get a quantitative assessment of cleaning effectiveness by the detergents. Additionally, washed fabrics are compared by visual inspection of the actual fabric, or an image of the fabric, to determine if the quantification is a perceivable effect. Often, these reflective measurements and visual assessments are complicated by the topography/construction of the fabric, fabric material composition, and color of the fabric. Such complications can make evaluation difficult, especially when there is a low amount of the residual stain or soil. Additionally, spectrophotometric measurements are slow and not very efficient.

Accordingly, it is desirable to have methods and systems that can analyze stained and soiled fabrics with an enhanced visual or quantitative assessment of stain and soil presence and, in particular, wherein such analysis can be done efficiently and quickly. Additionally, there is a need to be able to provide methods and systems that enable effective visual and/or quantitative assessments of stained fabric where the stain is invisible, or difficult to perceive, to the human eye or using conventional imaging systems. It is also desirable to be able to be able to measure and visualize soils that overlap on a fabric.

### SUMMARY OF THE DISCLOSURE

The present disclosure relates to methods for visualization and measurement of a soiling substance on a fabric. In many cases, fabric stains, also referred to herein as a soiling substance present on a fabric, can be hard to perceive due to lack of color and thus cannot be visualized effectively using conventional imaging systems, e.g., which use colorimetric and/or visible spectra. The present disclosure improves visualization and measurement of fabric stains through use of imaging devices that provide enhanced imaging of absorption and fluorescent emission signals produced by a signaling agent when illuminated by light of a defined wavelength or wavelength band.

Some aspects of the present disclosure provide methods of visualizing a soiling substance on a fabric. In some embodiments, methods according to the present disclosure may comprise providing a fabric that includes at least one soiling substance, applying a signaling agent to an area of the fabric that includes the at least one soiling substance, illuminating at least a portion of the area of the fabric that includes the at least one soiling substance and the signaling agent with light of a wavelength or wavelength band that is one or both of absorbed by the signaling agent or effective to cause fluorescent emission from the signaling agent, and detecting one or both of an absorbance signal and an emission signal from the signaling agent when illuminated. In further embodiments, the methods may be defined in relation to one or more of the following statements, which may be combined in any number or order.

The signaling agent may comprise a chromophore.

The signaling agent can be a porphyrin, for example, such as chlorophyll or vitamin B.

The signaling agent can be configured to bind to the at least one soiling agent.

The signaling agent can be configured to bind to one or more specified soiling agents. The methods may comprise applying at least a first signaling agent and a second signaling agent.

Different signaling agents, such as a first signaling agent and the second signaling agent, may exhibit one or both of different absorption spectrums and different fluorescent emission spectrums.

The methods may comprise illuminating with ultraviolet light.

The methods may comprise illuminating with light of a wavelength or wavelength band that is within the range of 100 nm to 800 nm.

The methods may comprise illuminating with light of a wavelength or wavelength band within the range of 200 nm to 600 nm.

The methods may comprise imaging the at least a portion of the area of the fabric that includes the at least one soiling substance and the signaling agent with a multispectral or hyperspectral camera.

The methods may comprise identifying a distribution of the at least one soiling substance on the fabric.

The methods may comprise quantifying an amount of the at least one soiling substance on the fabric based on an intensity of one or both of the absorbance signal and the emission signal from the signaling agent.

Detecting one or both of the absorbance signal and the emission signal from the signaling agent may comprise filtering through one or more filters of defined wavelengths or wavelength bands.

Defined wavelengths or wavelength bands used for filter may correspond to the wavelength or wavelength band that is one or both of absorbed by the signaling agent or effective to cause fluorescent emission from the signaling agent.

In another aspect, the present disclosure provides a method for quantifying efficacy of a composition for removal of a soiling substance from a fabric. Such methods may comprise, for example, treating a fabric that includes at least one soiling substance with a composition for removal of the at least one soiling substance, visualizing the at least one soiling substance on the fabric according to the method described herein above (e.g., according to the method of visualizing a soiling substance on a fabric as described herein) both before and after the treating, and calculating a percentage of stain removal based on a change in intensity of one or both of the absorbance signal and the emission signal from the signaling agent as detected before the treating and after the treating. In further embodiments, the methods may be defined in relation to one or more of the following statements, which may be combined in any number or order.

The composition can be a laundry detergent, and the treating can comprise laundering the fabric using the laundry detergent.

Laundering can be carried out in an automatic washing machine.

The composition can be a stain removal composition, and the treating can comprise applying the stain removal composition directly to fabric.

The methods may further comprise laundering the fabric with the stain removal composition applied thereto.

These and other features, aspects, and advantages of the present disclosure will be apparent from a reading of the following detailed description together with the accompanying drawings, which are briefly described below. The present disclosure includes any combination of two, three, four, or more features or elements set forth in this disclosure or recited in any one or more of the claims, regardless of whether such features or elements are expressly combined or otherwise recited in a specific embodiment description or claim herein. This disclosure is intended to be read holistically such that any separable features or elements of the disclosure, in any of its aspects and embodiments, should be viewed as intended to be combinable, unless the context of the disclosure clearly dictates otherwise.

### BRIEF DESCRIPTION OF THE FIGURES

Having thus described the disclosure in general terms, reference will now be made to the accompanying drawings, which are not necessarily drawn to scale, and wherein:

FIG. 1 is a flowchart illustrating a method of visualizing a soiling substance on a fabric, according to an example embodiment of the present disclosure;

FIG. 2 is a flowchart illustrating a method for quantifying efficacy of a composition for removal of a soiling substance from a fabric, according to an example embodiment of the present disclosure;

FIG. 3 illustrates hyperspectral images of various olive oil samples on a fabric, according to an example embodiment of the present disclosure;

FIG. 4A illustrates conventional images (e.g., view under white light) of six fabric samples including a soiling substance in varying amounts and a signaling agent, according to an example embodiment of the present disclosure;

FIG. 4B illustrates hyperspectral images of the six fabric provided in FIG. 4A, according to an example embodiment of the present disclosure;

FIG. 5A illustrates hyperspectral images of four fabric samples, including a soiling substance, present in varying amounts, that have been treated with a signaling agent, according to an example embodiment of the present disclosure;

FIG. 5B is a graph illustrating the fluorescent intensity versus wavelength of each fabric sample depicted in FIG. 5A;

FIG. 6A illustrates two hyperspectral images and two conventional images (e.g., viewed under white light) of a fabric sample, including a soiling substance and a signaling agent, both before and after washing, according to an example embodiment of the present disclosure;

FIG. 6B is a graph illustrating the fluorescent intensity versus wavelength of the fabric samples depicted in FIG. 6A both before and after washing in a conventional machine washer, according to an example embodiment of the present disclosure;

FIG. 7 is a graph illustrating the fluorescent intensity versus wavelength of four fabric samples, including varying soil loads and a signaling agent, according to an example embodiment of the present disclosure;

FIG. 8 illustrates hyperspectral images of the four fabric samples provided in FIG. 7, according to an example embodiment of the present disclosure;

FIG. 9 is a graph of the fluorescent intensity of several fabric samples including a soiling substance and a signaling agent, which have been treated using various different washing techniques, according to an example embodiment of the present disclosure; and

FIG. 10 illustrates hyperspectral images of fabric samples treated using treatment 1 and treatment 2 respectively, according to an example embodiment of the present disclosure.

### DETAILED DESCRIPTION OF THE DISCLOSURE

The present disclosure now will be described more fully hereinafter with reference to specific embodiments. Indeed, the disclosure may be embodied in many different forms and should not be construed as limited to the embodiments set forth herein; rather, these embodiments are provided so that this disclosure will satisfy applicable legal requirements. As used in the specification, and in the appended claims, the singular forms “a,” “an,” “the,” include plural referents unless the context clearly dictates otherwise.

Some aspects of the present disclosure relate to methods of visualizing stains or soiling substances on a fabric. As noted above, in many cases, fabric stains can be hard to perceive due to lack of color and thus cannot be visualized effectively using conventional imaging systems, e.g., which use colorimetric and/or visible spectra. For example, conventional imaging and colorimetric techniques use reflection for acquisition and analysis, which is a method that lacks a degree of precision. Incident light that is used to illuminate a sample is reflected to a detector and is greatly impacted by the optical properties of the sample including, but not limited to, color, topography, and material composition. The present disclosure overcomes these problems through use of imaging devices which provide enhanced imaging of absorption and fluorescent emission signals produced by a signaling agent in response to light of a defined wavelength or wavelength band and are, in general, much more selective and sensitive than conventional imaging. As such, it is possible to visualize multiple fluorescent emissions, or absorption signals, by selecting the appropriate signaling agent and the appropriate filter for wavelength along with the appropriate wavelength for stimulation.

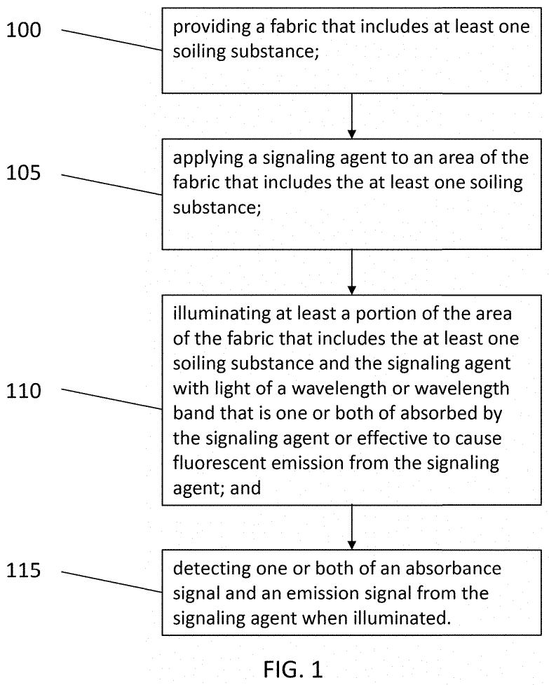

In some aspects, the present disclosure provides a method of visualizing a soiling substance on a fabric. As shown in FIG. 1, such methods include providing a fabric as described herein that includes at least one soiling substance at operation 100. The soiling substance is not intended to be limiting and it should be noted that the term “soiling substance” is intended to encompass any material (visible or invisible to the human eye) that is retained on the fabric and that was not originally a part of the fabric. A soiling substance may also be referenced as a staining substance or as simply “soil” or a “stain.” For example, a soiling substance may include, but is not limited to, grease, blood, dirt or debris, mud, grass, oils, dyes, food remnants, lipstick, inks, sweat or other bodily fluids, dead skin cells, beer, wine, juices, teas, coffee, other commonly used liquids, and the like.

A “fabric” as used herein, generally refers to any textile material formed from fibers. A fabric may particularly be a material formed by knitting, weaving, or stretching natural or synthetic fibers together. In some embodiments, non-woven items can also be included. For example, a fabric material as defined herein may comprise natural fibers and/or synthetic (i.e., man-made) fibers. Natural fibers can encompass cellulose-based fibers, such as bamboo, flax, hemp, jute, ramie, manila, sisal, cotton, and kapok, protein-based fibers, such as alpaca, camel, cashmere, llama, mohair, vicuna, wool, and silk, and mineral-based fibers, such as asbestos. Synthetic fibers can be organic or inorganic in origin. Organic, synthetic fibers includes those based on natural polymers, such as Rayon, Lyocell, Acetate, triacetate, azlon, and polylactic acid (PLA) and those based on synthetic polymers, such as acrylic, anidex, aramid, elastoester, fluoropolymer, lastrile, melamine, modacrylic, novoloid, nylon, nytril, olefin, polybenzimidazole (PBI), polyester, rubber, saran, spandex, sulfar, vinal, and vinyon. In some embodiments, fabrics according to the present disclosure may be in the form of clothing (e.g., shirt, pants, swimwear, etc.), linens (e.g., curtains, sheets, towels, etc.), upholstery (e.g., furniture, carpets, car interiors, etc.), cloths, felts, satins, lace, and the like.

As depicted in FIG. 1, at operation 105, the method of the present disclosure may comprise applying a signaling agent to an area of the fabric that includes the at least one soiling substance. Generally, the area of the fabric including the at least one soiling substance may vary in size and/or shape and/or positioning. In some embodiments, the area of the fabric including the soiling substance may include a single area on the fabric or multiple individual areas of the fabric. In still other embodiments, the area of the fabric including the at least one soiling substance may include more than one soiling substance, for example, such as at least 2, at least 3, at least 4, at least 5, at least 6, at least 7, at least 8, at least 9, or at least 10 soiling substances.

In some embodiments, the signaling agent applied to the fabric may vary. As used herein, a “signaling agent” generally refers to any compound effective to absorb light of a specified wavelength or wavelength band and/or effective to produce a fluorescent emission when contacted with light of a specified wavelength or wavelength band. In some embodiments, the signaling agent is a compound comprising a chromophore. As used herein, the term “chromophore” refers to an atom or group whose presence is responsible for the color of a particular compound, for example, a chromophore is the moiety that causes a conformational change of the molecule when hit by light at a certain wavelength. Such chromophores may be naturally occurring or synthetic and generally exhibit one or both of fluorescence and absorbance when contacted by light of a certain wavelength or wavelength band.

In some embodiments, the signaling agent is a porphyrin. Example porphyrins include, but are not limited to, chlorophyll and heme (e.g., an iron-containing compound of the porphyrin class which forms the non-protein part of hemoglobin and other biological molecules). However, the particular type of porphyrin used is not intended to be so limiting. For example, any porphyrin effective as a chromophore so as to exhibit fluorescence and/or absorbance when hit by light of a certain wavelength may be suitable. As used herein, a “porphyrin” generally refers to any naturally occurring heterocyclic, macrocycle organic compounds, composed of four modified pyrrole subunits interconnected at their a carbon atoms via methine bridges (═CH—). Porphyrins are typically pigments, and some have metal ions that are bound to the center porphyrin ring. In particular, porphyrins typically have a conjugated bonding structure which gives rise to the properties of electromagnetic energy absorption and fluorescent emission. In some embodiments, the outer ring of a porphyrin molecule can be chemically modified for desirable properties (for example, water or oil solubility). In some embodiments, porphyrins as described herein may be synthetic or natural in origin. Without intending to be bound by theory, it is expected that a wide range of porphyrins, having metal centers (e.g. Mg, Cu, Zn, Fe, etc.), or existing without metal centers, can be effective chromophores as long as they exhibit fluorescence and/or absorbance when illuminated under ultraviolet light.

Other potential active compounds or chromophores suitable for use may include species with structures similar to porphyrins, for example, vitamin B; vitamin B12, a cobalamin with a porphyrin-like structure; carotenoids, e.g., such as beta-carotene; carotenoid derivatives, e.g., such as vitamin A; and the like. It is noted that in some embodiments inorganic fluorescent species, such as quantum dots, which are semiconductors and which can accommodate electronic transitions characteristic of fluorescence, could also be employed as chromophores, as long as they are compatible with the stain/soil mixture.

As noted above, the signaling agent may be configured to bind to the at least one soiling agent when applied to the area of the fabric including the at least one soiling agent. In some embodiments, for example, the signaling agent may be configured to bind to one or more specified soiling agents. In some embodiments, multiple signaling agents may be used to bind to one or more specified soiling agents, for example, the signaling agents may be specifically selected based on their ability to exhibit fluorescence when hit with light of a certain wavelength or wavelength band. In some embodiments, methods according to the present disclosure may comprise applying a plurality of different signaling agents, such as at least a first signaling agent and a second signaling agent. Typically, the amount of different signaling agents used is not meant to be limiting. In some embodiments, for example, the method may comprise applying at least 2, at least 3, at least 4, at least 5, at least 6, at least 7, at least 8, at least 9, or at least 10 different signaling agents to the area of the fabric including the one or more soiling substances. Generally, multiple signaling agents may be selected based on their ability to exhibit one or both of different absorption spectrums and different fluorescent emission spectrums. For example, in some embodiments, the first signaling agent and the second signaling agent exhibit one or both of different absorption spectrums and different fluorescent emission spectrums.

In certain embodiments, the signaling agent may be mixed with the at least one soiling agent and does not necessarily need to react or bind with the at least one soiling agent. In such embodiments, the signaling agent may be selected based on its hydrophobicity characteristics in relation to the at least one soiling substance. For example, a signaling agent with a similar hydrophobicity to the at least one soiling substance may be used. In still other embodiments, the signaling agent may be reacted with the at least one soiling substance. Generally, it should be noted that the signaling agent can be detected so long as the absorption or fluorescent emission of the signaling agent is not blocked or obscured by the at least one soiling substance.

Generally, the amount of the signaling agent applied to the area of the fabric including the at least one soiling substance may vary. In some embodiments, the amount of the signaling agent is dependent on the type of chromophore used, the type and/or characteristics of the soiling substance, and the particular substrate/fabric being treated. For example, the amount of the signaling agent applied to the area of the fabric including the at least one soiling substance may be selected based on a threshold value of the overall detection system so as to avoid detector/sensor saturation. In some embodiments, for example, the signaling agent is applied to the area of the fabric including the at least one soiling substance in an amount in the range of about 1 ppb to about 10 ppm, about 100 ppb to about 10 ppm, or about 1 ppm to about 100 ppm, based on the total weight of the soiling substance. In some embodiments, the signaling agent is applied to the area of the fabric including the at least one soiling substance in an amount of at least about 1 ppb, at least about 50 ppb, at least about 100 ppb, at least about 1 ppm, at least about 5 ppm, or at least about 10 ppm.

Referring back to FIG. 1, as noted at operation 110, the methods according to the present disclosure may comprise illuminating at least a portion of the area of the fabric that includes the at least one soiling substance and the signaling agent with light of a wavelength or wavelength band that is one or both of absorbed by the signaling agent or effective to cause fluorescent emission from the signaling agent. The overall wavelength of the light used for illumination may vary based on the signaling agent used and/or the type of soiling substance. In some embodiments, the methods according to the disclosure may comprise illuminating at least a portion of the area of the fabric including the at least one soiling substance with ultraviolet light. In some embodiments, for example, the at least a portion of the area of the fabric including the at least one soiling substance may be illuminated with light of a wavelength or wavelength band that is within the range of about 1 nm to about 1000 nm, about 100 nm to about 800 nm, about 200 nm to about 600 nm, or about 400 nm to about 600 nm.

In some embodiments, the methods according to the present disclosure may comprise detecting one or both of an absorbance signal and an emission signal from the signaling agent when illuminated, for example, as depicted at operation 115 of FIG. 1. In some embodiments, the detecting may comprise imaging the at least a portion of the area of the fabric that includes the at least one soiling substance and the signaling agent with an imaging device. In certain embodiments, for example, the imaging device may be in the form of a multispectral or hyperspectral camera or imaging system. Multispectral and hyperspectral cameras have found use in many applications and industries. For example, some particular applications include dermatology, quality inspection, food processing, color inspection, crime scene investigation, fingerprint technology, drone technologies, surveillance systems, military applications, and process monitoring. Generally, multispectral and hyperspectral cameras are useful in a variety of applications due to their ability to illuminate a target with a specified wavelength and capture the entire electromagnetic spectrum. Using this type of imaging system, it is possible to illuminate a target with light of the chosen wavelength or wavelength band, make either absorption or fluorescent measurements, and acquire enhanced images based on the absorption and/or fluorescent measurements.

Examples of multispectral or hyperspectral imaging devices and their use in a variety of applications are described, for example, in U.S. Pat. No. 8,238,623 to Stephan et al.; and U.S. Pat. No. 9,551,616 to McQuilkin et al.; each of which are incorporated herein by reference in their entirety. Other examples of multispectral imaging systems and hyperspectral imaging systems are described, for example, in U.S. Pat. No. 8,174,694 to Bodkin; U.S. Pat. No. 8,351,045 to Mitchell et. el.; U.S. Pat. No. 9,692,991 to Zhang et al.; and U.S. Pat. No. 10,574,911 to Dvir; each of which are incorporated herein by reference in their entirety. Additionally, multispectral imaging systems and hyperspectral imaging systems are available as Spectracam® from Newtone Inc., Princeton, N.J., for example.

As used herein, a “multispectral camera” or “multispectral imaging system” generally refers to a camera or instrument effective to provide multispectral imaging, wherein multispectral imaging is defined as capturing image data across a range of wavelengths using less than 100 individual wavelength bands across the electromagnetic spectrum (e.g., typically 2 to 20 individual wavelength bands). As used herein, a “hyperspectral camera” or “hyperspectral imaging system” generally refers to a camera or instrument effective to provide hyperspectral imaging, wherein hyperspectral imaging is defined as capturing image data across a range of wavelengths using 100 or more individual wavelength bands across the electromagnetic spectrum (e.g., typically hundreds or thousands of individual wavelength bands having a range of about 10 to about 20 nm each). Typically, the individual wavelength bands may be separated by filters and/or detected via the use of instruments that are sensitive to particular wavelengths, including light from varying frequencies (e.g., such as visible light, infrared light, ultraviolet light, etc.). In some embodiments, for example, a multispectral or hyperspectral camera may be configured to capture image data across a wavelength range of about 1 nm to about 1000 nm, about 100 nm to about 800 nm, about 200 nm to about 600 nm, or about 400 nm to about 600 nm.

In some embodiments, the detecting one or both of the absorbance signal and the emission signal from the signaling agent when illuminated (e.g., as depicted at operation 115) may further comprise filtering through one or more filters of defined wavelengths or wavelength bands. In some embodiments, for example, the defined wavelengths or wavelength bands may correspond to the wavelength or wavelength band that is one or both of absorbed by the signaling agent or effective to cause fluorescent emission from the signaling agent. As noted above, the particular wavelengths or wavelength bands are not particularly limiting and the imaging devices as described herein may be configured to detect one or both of an absorbance signal and/or a fluorescent emission signal within a wavelength range of about 1 nm to about 1000 nm. In some embodiments, the imaging device (e.g., such as a multispectral camera or a hyperspectral camera or similar imaging device) may be configured to direct light of a specified wavelength or wavelength band to illuminate the area of the fabric including the signaling agent and the at least one soiling substance and the full visible spectral emission is captured for each pixel using a set of filters in front of the camera sensor. Such methods allow for the detection of both the absorbance signal and the fluorescent emission signal from the signaling agent when illuminated by the light. Typically, the amount of filters used to capture individual wavelengths or wavelength bands may vary based on the type of imaging device used. In some embodiments, the amount of filters employed may be at least 2, at least 5, at least 10, at least 20, at least 30, at least 40, or at least 50 filters, such as 2 to 100, 2 to 50, 5 to 50, 10 to 45, 20 to 45, 25 to 40, or 25 to 35 filters. In certain embodiments, a set of 31 filters may expressly be useful to achieve the desired imaging.

In some aspects, the methods according to the disclosure may comprise one or more additional steps. In some embodiments, the methods disclosed herein may further comprise identifying a distribution of the at least one soiling substance on the fabric. For example, the methods of the present disclosure can be used to identify the presence of one or multiple soiling substances, in one or more locations/areas on the fabric. In some embodiments, multiple signaling agents can be used and each signaling agent may produce a different absorbance signal and/or emission signal when illuminated by light of a certain wavelength. By using multiple wavelength filters, each individual absorbance and fluorescent emission signal can be detected by the imaging device and thereby identify the distribution of various soiling substances/signaling agents on the fabric.

In some embodiments, the methods may further comprise quantifying an amount of the at least one soiling substance on the fabric based on an intensity of one or both of the absorbance signal and the emission signal from the signaling agent. For example, where the absorbance signal and/or the emission signal detected is higher, the amount of the soiling substance present in the illuminated area is generally also higher. Likewise, where the absorbance signal and/or the emission signal detected is lower in a certain area, the amount of the soiling substance present in the illuminated area is generally lower, comparatively. Thus, the methods may comprise correlating a measured absorbance signal and/or emission signal to a concentration (e.g., mass, volume, or area) of a specific soiling substance. In some embodiments, mapping of soiling substances based on measured absorbance signal and/or emission signal may be carried out. For example, a lookup table may be generated so that a specific absorbance signal and/or emission signal for a specific soiling substance when illuminated by a light of a specific wavelength or wavelength band can be identified. When such lookup table is included with computer programming, the present methods can include obtaining an absorbance signal and/or emission signal as otherwise disclosed herein using instrumentation combined with the computer programming so that a specifically measured absorbance signal and/or emission signal can identify a specific soiling substance, and the intensity of the measured signal can identify a concentration of the specific soiling substance on the tested fabric.

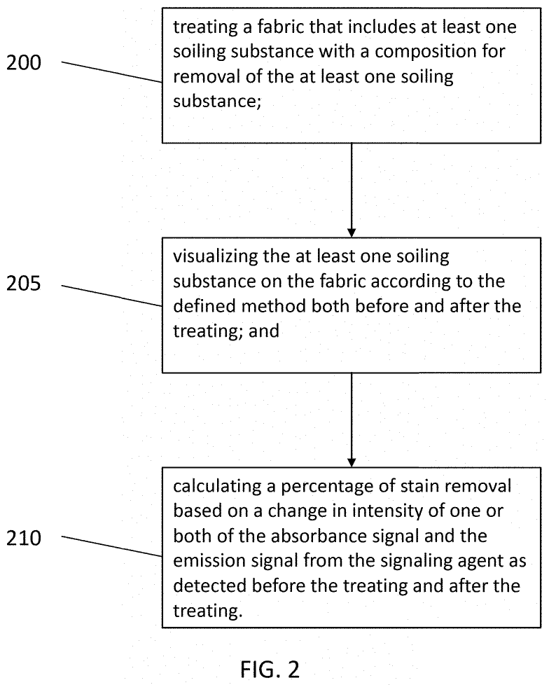

In some other aspects of the present disclosure are provided methods for quantifying efficacy of a composition for removal of a soiling substance from a fabric. For example, as noted above, the methods of the present disclosure can advantageously provide visualization of one or more soiling substances such that the area containing the soiling substance can be targeted during cleaning or treatment of the fabric. As depicted in FIG. 2, for example, such methods may comprise treating a fabric that includes at least one soiling substance with a composition for removal of the at least one soiling substance at operation 200. It should be noted that the fabric and the at least one soiling substance may include any fabric or soiling substance as defined herein above with respect to the methods of the present disclosure.

As noted herein the fabric may be treated with a composition for removal of the at least one soiling substance. The particular composition used is not intended to be limiting and may include any composition generally known in the art for laundering, cleaning, and/or removal of stains from a fabric material as described herein above. Non-limiting examples of compositions suitable for use in the above-identified method include, but are not limited to, powdered or liquid detergents, soaps, bleach, stain removers, laundry boosters, optical brighteners, fabric softeners, shampoos, water softeners, surfactants, baking soda, detoxifiers, disinfectants, sanitizers, hydrogen peroxide, whiteners, vinegar, odor removers, water, other consumer or commercial cleaning products, and the like. Such compositions may include compositions used in dry cleaning.

In some embodiments, the methods according to the present disclosure may optionally comprise laundering the fabric after treating the fabric with the composition. The type of laundering is not intending to be particularly limiting and may include, for example, any laundering method generally known in the art for laundering and/or cleaning fabrics. Non-limiting examples of laundering a fabric may include, but are not limited to, consumer or commercial machine washing, hand washing or rinsing, steam cleaning, dry cleaning, sponging, blotting, and the like. Typically, the laundering method and the type of treatment composition may vary based on the type of fabric and/or the composition of the soiling substance and/or based on the size or shape of the fabric. Likewise, the treatment composition may include one or more compositions as described herein and the fabric may undergo one or more laundering treatments as described herein. In certain embodiments, the composition is a laundry detergent, and the treating may comprise laundering the fabric using the laundry detergent. In such embodiments, the laundering is carried out in an automatic washing machine. In certain other embodiments, the composition is a stain removal composition, and the treating may comprise applying the stain removal composition directly to fabric. In such embodiments, the method may further comprise laundering the fabric with the stain removal composition applied thereto.

Referring back to FIG. 2, the methods according to the present disclosure may comprise visualizing the at least one soiling substance on the fabric both before and after the treating, for example as depicted at operation 205. Generally, it should be noted that by visualizing the soiling substance both before and after the treating it is possible to quantify the amount of the soiling substance that remains on the fabric following the treating and/or the amount of the soiling substance that was removed by the treatment. Advantageously, visualizing the stain both before and after the treating allows one to observe the effectiveness of the treatment composition in removing the soiling substance, for example, as will be discussed in more detail herein.

As depicted at operation 210 of FIG. 2, the methods of the present disclosure may comprise, in some embodiments, calculating a percentage of stain removal based on a change in intensity of one or both of the absorbance signal and the emission signal from the signaling agent as detected before the treating and after the treating. For example, the fluorescent intensity (I) can be used to give an estimate of the percentage stain removal (or presence thereof remaining on the fabric) using an equation analogous to the stain removal equation for detergency provided in ASTM D4265 (Standard Guide for Evaluating Stain Removal Performance in Home Laundering), for example, as provided herein below.

$$Stain Removal \% = 100 \times \frac{{I}_{u n w a s h e d} - {I}_{w a s h e d}}{{I}_{u n w a s h e d}}$$

### EXPERIMENTAL

Aspects of the present invention are more fully illustrated by the following examples, which are set forth to illustrate certain aspects of the present invention and are not to be construed as limiting thereof.

### Example 1

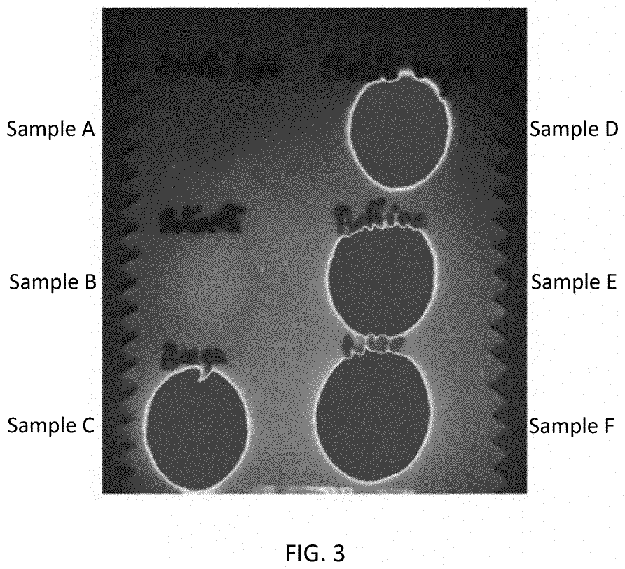

Testing was conducted to evaluate the absorbance and fluorescent emission properties of various consumer olive oil samples using a hyperspectral imaging camera. Two samples of refined, clear olive oil (e.g., Samples A and B) were illuminated under a hyperspectral camera and four samples of virgin olive oil (e.g., Samples C, D, E, and F) were illuminated under a hyperspectral camera. It should be noted that refined olive oil and virgin olive oil were selected for testing because virgin olive oil is generally known to contain about 1-10 ppm of chlorophyll while the refined, clear olive oil typically contains none. As shown in FIG. 3, the two samples of the refined, clear olive oil did not provide an absorbance or emission signal when illuminated, whereas the virgin olive oil samples, which contained chlorophyll, did provide an intense fluorescent emission signal when illuminated. Thus, it was determined that the presence of chlorophyll (e.g., a porphyrin) in the olive oil samples was effective for providing a detectable fluorescent emission signal.

### Example 2

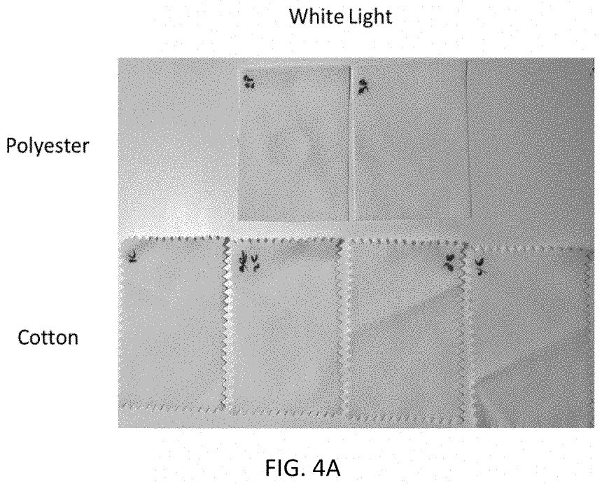

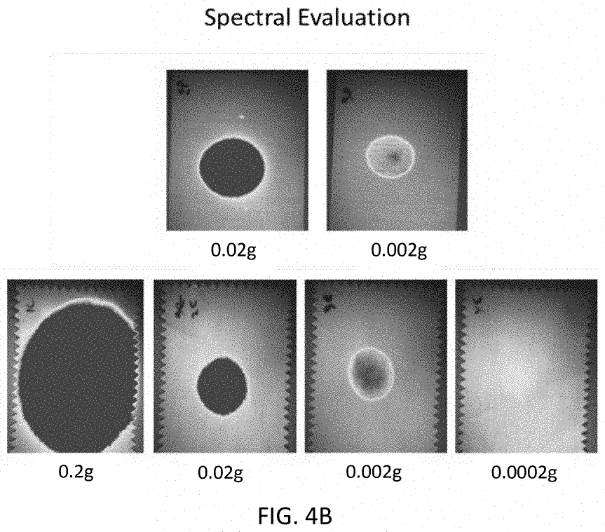

Six samples were prepared and evaluated under conventional imaging techniques (e.g., using visible light reflectance) and using the enhanced imaging techniques provided herein (e.g., viewing the absorbance and fluorescent emission signals via a hyperspectral camera). Two of the samples were prepared by applying an artificial sebum composition to a swatch of polyester fabric. Generally, the artificial sebum composition included a content of extra-virgin olive oil (e.g., known to contain about 30 ppm chlorophyll) as the signaling agent and a content of a vaseline/squalene mixture as the soiling substance. In particular, the artificial sebum composition included about 60% by weight extra-virgin olive oil, about 26% by weight Vaseline, and about 14% by weight squalene. Approximately 0.02 g of the artificial sebum composition was applied to the first sample and approximately 0.002 g of the artificial sebum composition was applied to the second sample. Next, the polyester fabric samples were viewed using conventional imaging (under white light) and using hyperspectral imaging to evaluate the visibility of the stain on the fabric. As depicted in FIG. 4A, the soiling substance (even with the signaling agent present) is completely invisible to the naked eye using conventional imaging. However, as depicted in FIG. 4B the area of the samples including the soiling substance was clearly identified when evaluated using hyperspectral imaging.

Next, four more samples were prepared by applying an artificial sebum composition to a swatch of cotton fabric. Approximately 0.2 g of the artificial sebum composition was applied to the first sample; approximately 0.02 g of the artificial sebum composition was applied to the second sample; approximately 0.002 g of the artificial sebum composition was applied to the second sample; approximately 0.0002 g of the artificial sebum composition was applied to the second sample. Next, the area containing the artificial sebum composition was treated with an amount of signaling agent (e.g., including a porphyrin). Then, the cotton fabric samples were viewed using conventional imaging (under white light) and using hyperspectral imaging to evaluate the visibility of the stain on the fabric. As depicted in FIG. 4A, the soiling substance (even with the signaling agent present) is completely invisible to the naked eye for each of the samples using conventional imaging. However, as depicted in FIG. 4B, the area of the samples including the soiling substance was clearly identified when evaluated using hyperspectral imaging. It is notable that the sample treated with only 0.0002 g of the artificial sebum composition was barely visible, and the visibility of the illuminated soiling substance with the applied signaling agent substantially proportionally decreased with the decreasing amount of the soiling substance that was applied. This indicates that the methods were not only effective for qualitative measurements (i.e., simply identifying the presence of the soiling substance) but were also effective for quantitative measurements (i.e., identifying the concentration of the soiling substance through correlation to signal intensity).

### Example 3

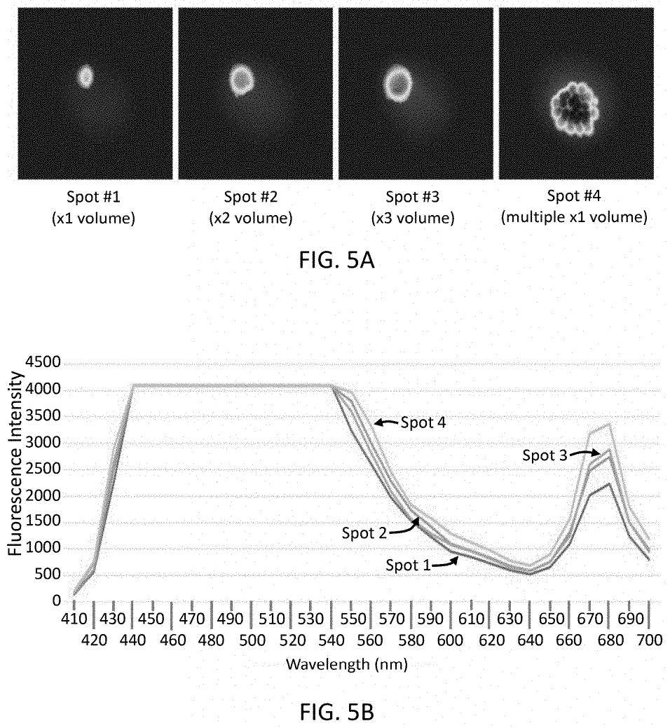

FIG. 5A depicts hyperspectral images of four fabric samples including a soiling substance, present in varying amounts, that have been treated with a signaling agent and evaluated using hyperspectral imaging. In particular, spot 1 includes the soiling substance and has been treated with a porphyrin; spot 2 includes the soiling substance at 2× the volume of spot 1 and has been treated with a porphyrin; spot 3 includes the soiling substance at 3× the volume of spot 1 and has been treated with a porphyrin; and spot 4 includes multiple individual droplets of the soiling substance and has been treated with a porphyrin. As depicted in FIG. 5, stains of varying amounts can be visualized on the fabric and differentiated based on the intensity of fluorescent emission. In addition, use of hyperspectral imaging can differentiate a larger stain spot from multiple smaller stain spots, for example as depicted with spot 4.

FIG. 5B is a graph illustrating the fluorescent intensity versus wavelength of each fabric sample depicted in FIG. 5A. As shown in FIG. 5, the higher the volume of the soiling substance, the higher the intensity of the fluorescent emission signal emitted therefrom and detected by the hyperspectral camera.

### Example 4

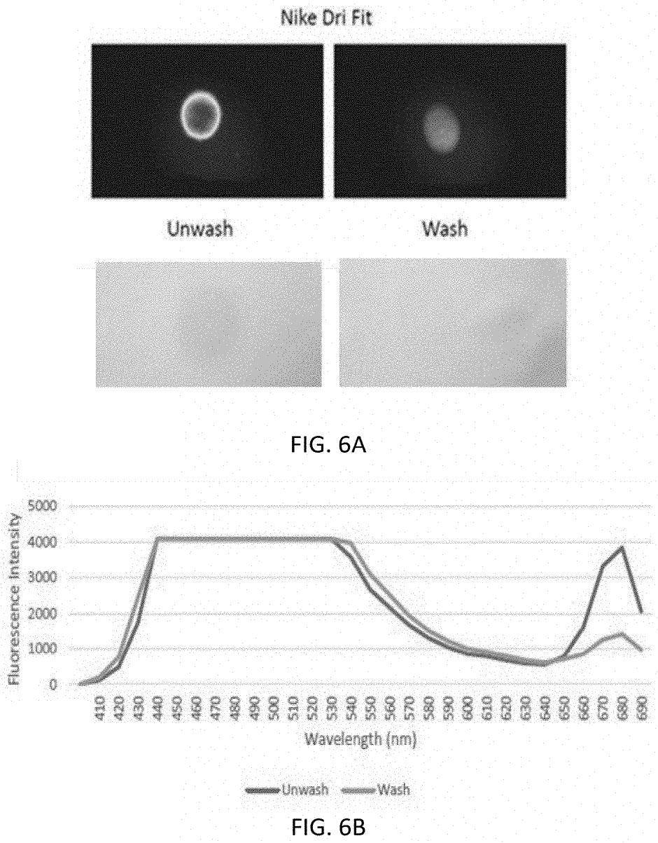

FIG. 6A depicts a two hyperspectral images and two conventional images (e.g., viewed under white light) of a fabric sample, including a soiling substance and a signaling agent, both before and after washing in a conventional machine washer. As shown in FIG. 6A, the soiling substance is almost completely invisible to the naked eye, whereas it is clearly visible both before and after washing when evaluated using hyperspectral imaging.

FIG. 6B is a graph illustrating the fluorescent intensity versus wavelength of the fabric samples depicted in FIG. 6A both before and after washing in a conventional machine washer. As shown in FIG. 6B, the intensity of the fluorescent emission signal is much higher for the fabric sample prior to washing. As noted herein above, the fluorescent intensity before and after washing can be used to calculate the percentage stain removal achieved during washing. For example, using the stain removal equation for detergency provided in ASTM D4265, the percentage stain removal can be calculated as follows.

$$Stain Removal \% = 100 \times \frac{Iunwashe d ( 4000 ) - Iwashe d ( 1500 )}{Iunwashe d ( 4000 )} = 62.5 \%$$

Therefore, washing of the fabric sample removed approximately 62.5% of the stain.

### Example 5

Testing was conducted to evaluate the effect of different soil loads on the fluorescent intensity as measured at a wavelength of about 680 nm. Initially, four fabric samples were prepared. First, a control sample was prepared by treating a polyester fabric with 100% virgin olive oil, which contained no soiling substance. Next, three samples were prepared by treating three different polyester fabrics with a defined amount of a model sebum composition, which included 60% virgin olive oil, 26% Vaseline, and 14% Squalene. In the instant example, the virgin olive oil in the sebum composition functioned as the signaling agent, whereas the Vaseline and Squalene functioned as the soiling composition for the purposes of this and other studies described herein below. Table 1 below shows the composition of the model sebum composition.

TABLE 1

| Ingredient | Percent Composition (%) |
|---|---|
| Extra Virgin Olive Oil | 60 |
| Vaseline | 26 |
| Squalene | 14 |

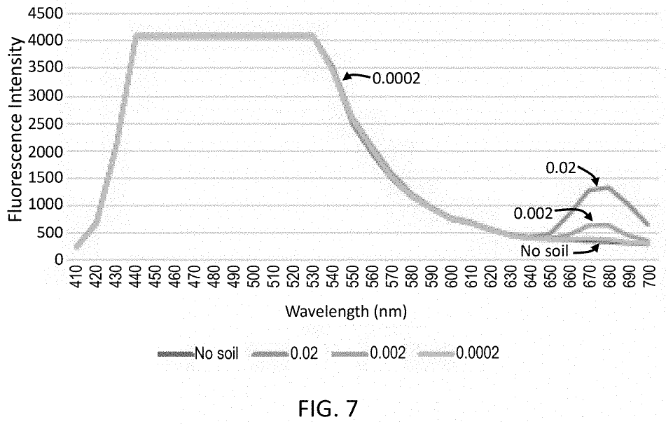

The first of the three treated samples was treated with 0.02 g of the model sebum composition. The second of the three treated samples was treated with 0.002 g of the model sebum composition. The third of the three treated samples was treated with 0.002 g of the model sebum composition. The fluorescent intensity spectra evaluated at a wavelength of 680 nm for the three treated samples and the control sample are shown in FIG. 7. As shown in FIG. 7, the fluorescent intensity increased exponentially as the soil load was increased.

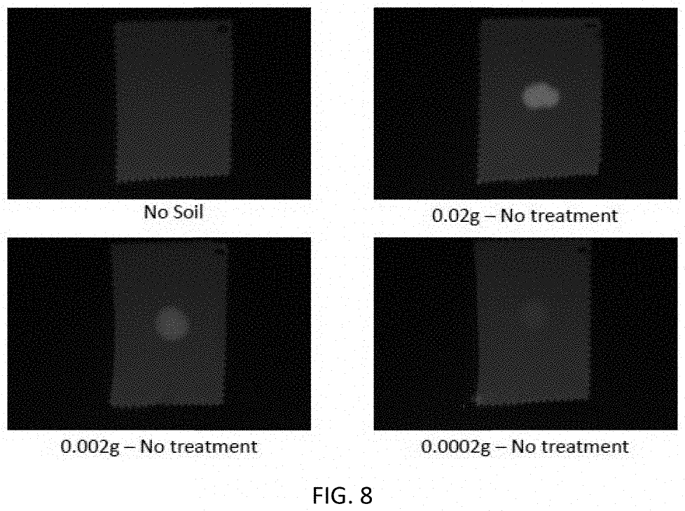

Next, each of the fabric samples were illuminated at 385 nm and imaged with a multispectral camera in order to directly image the soil spots. It is noted that the soiling substance was not easily discernible when viewed without the special imaging but became clearly visible when viewed with the UV illumination and multispectral camera. Hyperspectral images of each of the fabric samples are shown in FIG. 8. As shown in FIG. 8, the fluorescent intensity, and thereby the visibility of the stain, in the hyperspectral images increased qualitatively with the soil load.

### Example 6

Testing was conducted to evaluate the effect of different washing treatments on the fluorescent intensity as measured at a wavelength of about 680 nm. Initially, four identical polyester fabric samples were prepared by treating the fabric samples with the same sebum composition used in Example 5/Table 1 at a loading of 0.02 g. Next, each of the four samples were subjected to a different wash treatment. Control sample 1 did not include a wash treatment. Sample 4 was treated with commercial composition 1 and allowed to soak for 3 hours, followed by washing in a conventional machine washer. Sample 10 was washed with commercial composition 2 in a conventional machine washer. Finally, sample 16 was simply washed in water in a conventional machine washer. Table 2 below shows the sebum load for each sample and the washing treatment used.

TABLE 2

| Sample | Sebum | Treatment | |
|---|---|---|---|
| Number | load (g) | number | Wash treatment |
| 1 | 0.02 | | None |
| 4 | 0.02 | 1 | Commercial Composition 1 |
| 10 | 0.02 | 2 | Commercial Composition 2 |
| 16 | 0.02 | 3 | Water wash |

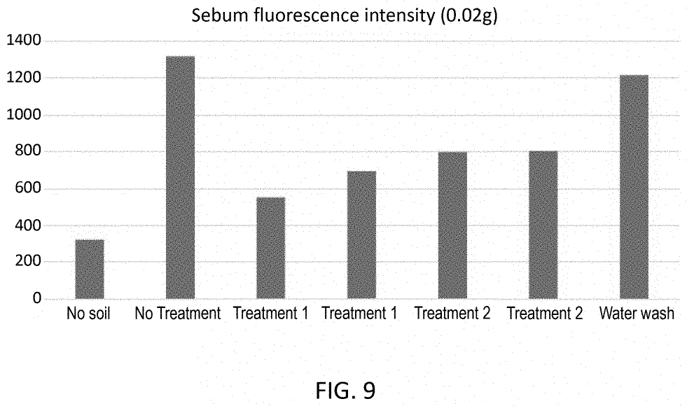

The fluorescent intensity spectra evaluated at a wavelength of 680 nm for the three fabric samples and the control sample are shown in FIG. 9. As shown in FIG. 9, treatments 1 and 2 yielded lower fluorescence levels than the control sample and the water wash indicating improved soil removal for both treatments. Further, treatment 1 yielded lower fluorescence levels than treatment 2 indicating improved soil removal for treatment 1 as compared to treatment 2.

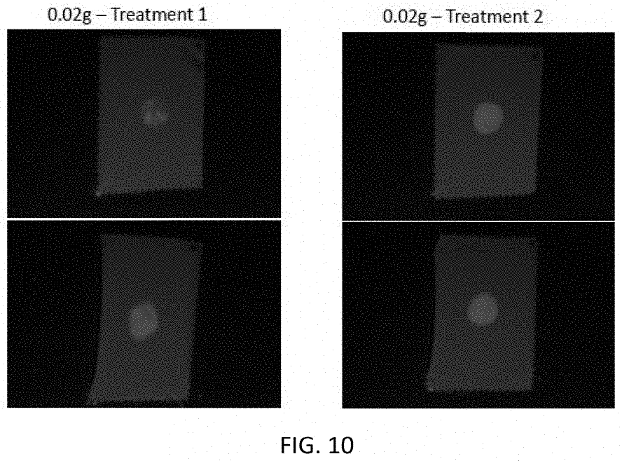

Next, samples 4 and 10 (having been subjected to treatments 1 and 2, respectively) were illuminated at 385 nm and imaged with a multispectral camera in order to directly image the soil spots. Two replicate hyperspectral images of each of the fabric samples are shown in FIG. 10. As shown in FIG. 10, sample 4 exhibited visually lower degrees of color density/fluorescent intensity as compared to sample 10, indicating improved cleaning efficacy for treatment 1.

The terms “about” or “substantially” as used herein can indicate that certain recited values or conditions are intended to be read as encompassing the expressly recited value or condition and also values that are relatively close thereto or conditions that are recognized as being relatively close thereto. For example, unless otherwise indicated herein, a value of “about” a certain number or “substantially” a certain value can indicate the specific number or value as well as numbers or values that vary therefrom (+ or −) by 5% or less, 4% or less, 3% or less, 2% or less, or 1% or less. Similarly, unless otherwise indicated herein, a condition that substantially exists can indicate the condition is met exactly as described or claimed or is within typical manufacturing tolerances or would appear to meet the required condition upon casual observation even if not perfectly meeting the required condition. In some embodiments, the values or conditions may be defined as being express and, as such, the term “about” or “substantially” (and thus the noted variances) may be excluded from the express value.

Many modifications and other embodiments of the disclosure set forth herein will come to mind to one skilled in the art to which these disclosures pertain having the benefit of the teachings presented in the foregoing descriptions. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A method of visualizing a soiling substance on a fabric, the method comprising:
providing a fabric that includes at least one soiling substance;
applying a signaling agent to an area of the fabric that includes the at least one soiling sub stance;
illuminating at least a portion of the area of the fabric that includes the at least one soiling substance and the signaling agent with light of a wavelength or wavelength band that is one or both of absorbed by the signaling agent or effective to cause fluorescent emission from the signaling agent; and
detecting one or both of an absorbance signal and an emission signal from the signaling agent when illuminated.

2. The method of claim 1, wherein the signaling agent comprises a chromophore.

3. The method of claim 1, wherein the signaling agent is a porphyrin.

4. The method of claim 1, wherein the signaling agent is configured to bind to the at least one soiling agent.

5. The method of claim 1, wherein the signaling agent is configured to bind to one or more specified soiling agents.

6. The method of claim 1, comprising applying at least a first signaling agent and a second signaling agent.

7. The method of claim 6, wherein the first signaling agent and the second signaling agent exhibit one or both of different absorption spectrums and different fluorescent emission spectrums.

8. The method of claim 1, comprising illuminating with ultraviolet light.

9. The method of claim 1, comprising illuminating with light of a wavelength or wavelength band that is within the range of 100 nm to 800 nm.

10. The method of claim 9, wherein the wavelength or wavelength band is within the range of 200 nm to 600 nm.

11. The method of claim 1, wherein the detecting comprises imaging the at least a portion of the area of the fabric that includes the at least one soiling substance and the signaling agent with a multispectral or hyperspectral camera.

12. The method of claim 1, further comprising identifying a distribution of the at least one soiling substance on the fabric.

13. The method of claim 1, further comprising quantifying an amount of the at least one soiling substance on the fabric based on an intensity of one or both of the absorbance signal and the emission signal from the signaling agent.

14. The method of claim 1, wherein the detecting one or both of the absorbance signal and the emission signal from the signaling agent comprises filtering through one or more filters of defined wavelengths or wavelength bands.

15. The method of claim 14, wherein the defined wavelengths or wavelength bands correspond to the wavelength or wavelength band that is one or both of absorbed by the signaling agent or effective to cause fluorescent emission from the signaling agent.

16. A method for quantifying efficacy of a composition for removal of a soiling substance from a fabric, the method comprising:
treating a fabric that includes at least one soiling substance with a composition for removal of the at least one soiling substance;
visualizing the at least one soiling substance on the fabric according to the method of claim 1 both before and after the treating; and
calculating a percentage of stain removal based on a change in intensity of one or both of the absorbance signal and the emission signal from the signaling agent as detected before the treating and after the treating.

17. The method of claim 16, wherein the composition is a laundry detergent, and the treating comprises laundering the fabric using the laundry detergent.

18. The method of claim 17, wherein the laundering is carried out in an automatic washing machine.

19. The method of claim 16, wherein the composition is a stain removal composition, and the treating comprises applying the stain removal composition directly to fabric.

20. The method of claim 19, further comprising laundering the fabric with the stain removal composition applied thereto.
